(19) United States
(12) Patent Application Publication
(10) Pub. No.: US 2019/0083365 A1
(43) Pub. Date: Mar. 21, 2019

(54) MICRO-EMULSION CONCENTRATES WITH AMPHOTERIC SURFACTANTS

(71) Applicant: BASF SE, Ludwigshafen am Rhein (DE)

(72) Inventors: Matthias Hloucha, Düsseldorf-Holthausen (DE); Stefanie Seidler, Düsseldorf-Houlthausen (DE); Esther Kuesters, Düsseldorf-Holthausen (SE); Petra Schulte, Düsseldorf-Holthausen (DE); Gabriele Strauss, Düsseldorf--Holthausen (DE)

(21) Appl. No.: 16/099,453

(22) PCT Filed: May 2, 2017
(86) PCT No.: PCT/EP2017/060323

§ 371 Date: Nov. 7, 2018

(30) Foreign Application Priority Data
May 10, 2016 (EP) 16168939.3

(51) Int. Cl. A61K 8/02; A61K 8/06; A61K 8/34; A61K 8/37; A61K 8/44; A61K 8/60; A61Q 19/10

(52) U.S. Cl. CPC A61K 8/0208; A61K 8/068; A61K 8/345; A61K 2800/596; A61K 8/442; A61K 8/604; A61Q 19/10; A61K 8/375

(57) ABSTRACT

The invention is in the field of cosmetic compositions and relates to highly concentrated microemulsion concentrates with low viscosity, comprising specific surfactants and oils in selected quantitative ratios, which can be readily diluted with water, and also to the use of these dilutions for impregnating textile and paper for cosmetics.

### FIELD OF THE INVENTION

The invention relates to the field of cosmetic compositions in the form of low-viscosity microemulsion concentrates which, after dilution, are suitable for impregnating textile and paper for cosmetics, and also to the preparation of same.

### PRIOR ART

Sheet-like articles based on textile and/or paper have found a broad range of uses as cosmetic wipes, baby cleaning wipes, etc., commonly also referred to as wet wipes, which must satisfy the high, and also contradictory, demands of consumers. Thus, on the one hand, the compositions applied to the sheet-like articles must afford good cleaning performance, but also, on the other hand, must care for and condition the skin, and, especially if the compositions remain on the skin, must be extremely skin-friendly and free from constituents which irritate the skin.

Further requirements arise from the manufacturers of such sheet-like articles. Thus, manufacturers desire compositions that are as highly concentrated as possible in order to save on storage and transport costs, and at the same time these highly-concentrated compositions should be transparent and have low viscosity and, above all else, should remain at the applied concentration range during dilution with water, without gelling occurring. Subsequently, the aqueous dilutions themselves should have low viscosity and be sprayable. Furthermore, manufacturers expect high shelf life at low and high temperatures, both from the cosmetic concentrated compositions and also from the dilutions thereof, which high shelf life additionally should be afforded independently of perfume and preservative.

Microemulsions are macroscopically homogeneous, optically transparent mixtures of two mutually immiscible liquids. These are generally thermodynamically stable emulsions of oil and water that are prepared in the presence of selected emulsifiers in selected quantities.

The mean particle sizes of microemulsions are customarily less than 100 nm and have high transparency, as a result of which they differ from customary oil-in-water emulsions.

European patent application EP 1813251 describes cold-preparable microemulsion-like concentrates comprising mixtures of a nonionic emulsifier and a secondary emulsifier having optionally neutralized acid functions and also a cosurfactant and one or more oils. The microemulsion concentrates obtained are clear to transparent and have a total water content of greater than or equal to 15 to 60 wt %. Neither the concentrates nor the aqueous dilutions envisage the addition of further surfactants.

International patent application WO 98/24409 discloses foaming body cleansing compositions which, in addition to skin-compatible anionic surfactants (A), consist of alkyl polyglycosides (B) and a combination of a zwitterionic surfactant (C) and an ampholytic surfactant (D) for improving the foaming properties and the viscosity.

International patent application WO 02/072052 (EP1372599) describes emulsions that may be in diluted form for use on wipes. The emulsifiers are ethoxylated compounds and the oils are mineral oils which, however, are not desired by consumers of “green” cosmetics. In the dilutions of the emulsions, betaines are used as zwitterionic surfactants.

International patent application WO 03/066017 discloses washing preparations having plant oils, alkyl polyglycosides and amphoteric surfactants, that are used as shower oils. Such shower oils become milky white on addition of water. They are not dear, transparent microemulsions, and the aqueous dilutions do not lead to dear, transparent emulsions.

International application WO 2008/155075 discloses cosmetic preparations which comprise, in addition to non-alkoxylated surfactants selected from the group of anionic and zwitterionic or ampholytic surfactants, a microemulsion and at least one cationic polymer. These preparations are used as conditioning agent in shampoo and haft treatment compositions. For a better conditioning effect, a cationic polymer is necessary. In the examples, only shampoos are disclosed, which comprise cocamidopropyl betaine and a microemulsion in addition to ethoxylated anionic surfactants. Such shampoos comprise a high proportion of foaming surfactants and therefore are not considered as compositions for impregnating wipes and paper in the cosmetics industry. Furthermore, the preparations have a significantly higher proportion of water both in the microemulsion and in the ready-to-use hair shampoo, and so there are no concentrates present.

The object of the present invention consisted in providing concentrated compositions for impregnating textile and paper, which
as a concentrate having a water content of less than 35, preferably 25 wt %, are transparent and have low viscosity,
as a concentrate, are outstandingly storage-stable across a broad temperature range,
only comprise a small amount of, if any, ethoxylated compounds,
require no, or only small quantities, of preservatives,
tolerate different preservatives and perfume oils without this affecting the shelf life,
comprise skin-friendly low-foaming surfactants in the concentrate,
can be readily diluted with water without causing gelling or disrupting the emulsion,
can be diluted with water at room temperature so that the customer does not have to warm the emulsion,
are storage-stable, after dilution with water for impregnating the sheet-like articles, under fluctuating thermal stresses,
after dilution with water, have a very low viscosity, with viscosities of less than 400 or even less than 200 mPas,
wet textile and paper well in the dilution.

On use, the impregnated sheet-like articles made of textile and paper should produce a mild cleaning performance and a pleasant skin feel for the consumer. Furthermore, the impregnated sheet-like articles should retain moisture for a long time even after opening, so that the consumer can use a moist, and not dried-out, sheet-like article.

Surprisingly, it was found that concentrated microemulsions having selected emulsifiers in combination with a specific zwitterionic or amphoteric surfactant meet the complex requirements.

Microemulsion concentrates for impregnating textile and paper in cosmetics, comprising
a1) alkyl and/or alkenyl glycosides
a2) cosurfactants selected from the group formed of monoesters of glycerol with a C₆-C₂₂ fatty acid
a3) oils different to a2)
a4) amphoteric or zwitterionic surfactant selected from the group of amphoacetates in quantities of 4.75 to 6.5 wt % —based on concentrate—and
a5) water,

wherein the total water content is 0.5 to 35 wt %—based on concentrate.

Further subjects of the invention relate to the use of the alkyl and/or alkenyl glycosides and amphoacetates as emulsifier and/or surfactant mixture for preparing such microemulsion concentrates, a method for preparing such microemulsion concentrates, the use of same in the form of the aqueous dilutions thereof as impregnating agent for preparing sheet-like articles made of textile and paper in cosmetics, especially what are referred to as wet wipes, and also the impregnating agent itself.

The microemulsion concentrates according to the present teaching are preferably of the oil-in-water (O/W) type or have a bicontinuous structure. The microemulsion concentrates preferably comprise, as continuous phase, water (a5), at least one alkyl and/or alkenyl (oligo)glycoside (a1), an amphoteric or zwitterionic surfactant from the group of amphoacetates (a4) and a cosurfactant (a2) that differs therefrom, and also at least one water-insoluble oil (a3) as inner phase.

Microemulsion concentrates are macroscopically homogeneous, optically transparent, low-viscosity, thermodynamically stable mixtures.

The mean particle sizes are customarily less than 100 nm, preferably between 3 and 100 nm, determined according to the DLS method using an apparatus with the name Horiba LB-500. They have high transparency and are stable, upon centrifugation at 2000 rpm for at least 30 minutes, against visible phase separation.

The conductivity of the inventive microemulsion concentrates is preferably in the range of greater than/equal to 500 μSi/cm and particularly preferably greater than/equal to 1000 μSi/cm.

The transparency was measured as FNU Formazin Nephelometric Units—scattered light measurement (90° angle) according to the provisions of standard ISO 7027 with formazin as turbidity standard liquid. For the purposes of the invention, a preparation up to at most 200 FNU, measured according to the standard, is designated transparent.

In the present application, the term “low viscosity” represents viscosities below 500 mPas measured according to Brookfield/RVT/23° C.+/−3° C./Sp 1/20 rpm. They preferably have viscosities in the range from 1 to 400 mPas determined according to Brookfield/ RVT/23° C.+/−3° C./Sp 1/20 rpm.

The inventive, so-called oils a3) are organic, water-insoluble oil components that are liquid at room temperature; this means that they have a melting point lower than the customary room temperature of approximately 18 to 25° C.

For the purposes of the present invention, oils are understood to mean water-insoluble oil components that have a solubility in water at 20° C. of less than or equal to 2 wt %.

The terms “oils” and “water-insoluble oil components” are used synonymously.

The terms “amphoteric” and “zwitterionic” surfactants are also used synonymously and designate, as is generally customary, surfactants that have both a negatively-charged and a positively-charged functional group, or which are converted into same by adjusting the pH. The surface-active nature arises by the structure of the nonpolar and polar part.

a1) Alkyl and/or Alkenyl Oligoglycosides

The microemulsion concentrates necessarily comprise a1) alkyl and/or alkenyl oligoglycosides (hereinafter also referred to as “APGs”) as surfactants.

For the purposes of the present teaching, alkyl and/or alkenyl oligoglucosides preferably have the formula (I),

R¹O-[G]ₚ (I)

in which R¹ represents an alkyl and/or alkenyl radical having 4 to 22 carbon atoms, G represents a sugar radical having 5 or 6 carbon atoms and p represents numbers from 1 to 10. They can be obtained by the relevant methods of preparative organic chemistry. The alkyl and/or alkenyl oligoglycosides can be derived from aldoses or ketoses having 5 or 6 carbon atoms, preferably from glucose. The preferred alkyl and/or alkenyl oligoglycosides are therefore alkyl and/or alkenyl oligoglucosides. The index number p in the general formula (I) specifies the degree of oligomerization (DP), i.e. the distribution of mono- and oligoglycosides, and represents a number between 1 and 10. Whereas p in a given compound must always be an integer and can here in particular assume the values p=1 to 6, the value p for a particular alkyl oligoglycoside is an analytically determined calculated parameter which in most cases is a fraction. Preference is given to using alkyl and/or alkenyl oligoglycosides having a mean degree of oligomerization p of 1.1 to 3.0. From a technical applications perspective, preference is given to those alkyl and/or alkenyl oligoglycosides for which the degree of oligomerization is greater than 1.1 and less than 1.7 and is especially between 1.2 and 1.4.

Accordingly, in one embodiment the alkyl and/or alkenyl radical R¹ is derived from primary higher alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and also technical grade mixtures thereof which may be obtained as described above. Preference is given to alkyl oligoglucosides based on hydrogenated C_{12/14} coconut fatty alcohol.

The alkyl oligoglucoside sold by BASF under the trade name Plantacare® 1200 UP (INCI: Lauryl Glucoside) is outstanding.

For the purposes of the present teaching, the microemulsion concentrates comprise the components (a1) preferably in quantities of 5 to 20 wt %, preferably in quantities of 7 to 12 wt %—based on the total weight of the microemulsion concentrates.

a2) Cosurfactant: Monoesters of Glycerol

For the purposes of the present invention, the inventive microemulsion concentrates additionally comprise, as cosurfactant (a2), monoesters of glycerol with a C₆-C₂₂ fatty acid.

These are preferably monoesters of glycerol of C₆-C₂₂ fatty acid mixtures that comprise unsaturated C₁₂-C₂₂ fatty acids. For the purposes of the invention, particular preference is given to glyceryl monoesters of unsaturated straight-chain C₁₂-C₂₂ fatty acids, and glyceryl monooleate is especially suitable. Both the pure glyceryl monooleate and the technical grades thereof are suitable for the purposes of the invention. A technical product is for example Monomuls® 90-O 18, a commercial product from BASF Personal Care & Nutrition GmbH.

For the purposes of the present invention, the monoesters of glycerol as cosurfactant a2) are preferably present in quantities of 3 to 20 wt %, preferably of 3 to 7 wt %—based on microemulsion concentrate.

a3) Oils

As necessary constituent a3), oils are present that preferably have a polarity of between 5 and 60 mN/m. The interfacial tension is determined as interfacial tension in mN/m, analogously to the ASTM method D971-99a (2004). The oils a3) are other than a2).

Suitable compounds a3) are for example selected from the group formed of
Guerbet alcohols based on fatty alcohols having 6 to 18 carbon atoms,
esters of linear C₆-C₂₂ fatty acids with linear C₆-C₂₂ fatty alcohols,
esters of linear C₆-C₂₂ fatty alcohols with branched C₆-C₃₂ carboxylic acids,
esters of branched C₃-C₁₈ alcohols with linear C₆-C₃₂ fatty acids,
esters of branched C₃-C₁₈ alcohols with branched C₆-C₃₂ carboxylic acids,
triglycerides based on C₆-C₁₄ fatty acids,
esters of C₂-C₁₂ dicarboxylic acids with linear alcohols having 1 to 22 carbon atoms or polyols having 2 to 10 carbon atoms and 2 to 6 hydroxyl groups,
vegetable oils,
linear or branched C₆-C₂₂ dialkyl carbonates,
linear or branched, symmetrical and/or asymmetrical dialkyl ethers having 6 to 22 carbon atoms per alkyl group,
“CX hydrocarbons” of the hydrocarbon group containing C7, C9, C11, C13, C15, C17, C19, C21 and/or C23 hydrocarbons.

Examples of the group of esters of linear C₆-C₂₂ fatty acids with linear C₆-C₂₂ fatty alcohols are caprylyl caprylate, myristyl myristate, myristyl palmitate, myristyl stearate, myristyl oleate, myristyl behenate, myristyl erucate, cetyl myristate, cetyl palmitate, cetyl stearate, cetyl oleate, cetyl behenate, cetyl erucate, stearyl myristate, stearyl palmitate, stearyl stearate, stearyl oleate, stearyl behenate, stearyl erucate, oleyl myristate, oleyl palmitate, oleyl stearate, oleyl oleate, oleyl behenate, oleyl erucate, behenyl myristate, behenyl palmitate, behenyl stearate, behenyl oleate, behenyl behenate, behenyl erucate, erucyl myristate, erucyl palmitate, erucyl stearate, erucyl oleate, erucyl behenate and erucyl erucate or mixtures thereof.

Examples of the group of esters of linear C₆-C₂₂ fatty alcohols with branched C₆-C₃₂ carboxylic acids are myristyl isostearate, cetyl isostearate, cetyl isononate, stearyl isononate, stearyl isostearate, cetyl isostearate, behenyl isostearate and erucyl isostearate or mixtures thereof. Particular preference is given here to cetyl isononate and/or stearyl isononate, i.e. isononanoic esters of cetyl and/or stearyl alcohol, such as Cetiol® SN, a commercial product from BASF Personal Care Nutrition GmbH.

Examples of the group of esters of branched C₃-C₁₈ alcohols with linear C₆-C₃₂ fatty acids are isostearyl myristate, isostearyl palmitate, isostearyl stearate, isostearyl oleate, isostearyl behenate, isopropyl palmitate, isopropyl stearate, ethylhexyl palmitate, ethylhexyl stearate or mixtures thereof.

An example of esters of branched C₃-C₁₈ alcohols with branched C₆-C₃₂ carboxylic acids is isostearyl isostearate.

Especially suitable as triglycerides are glycerol esters of fatty acids having 8 and/or 10 carbon atoms, wherein the triglyceride esters may also comprise proportions of diglyceride esters, for example triglyceride/diglyceride ester as can be obtained under the trade name Myritol® 312 from BASF Personal Care & Nutrition GmbH (INCI Name: Caprylic/Capric Triglyceride).

Dioctyl malate is for example a suitable dicarboxylic ester.

Further suitable are vegetable oils such as groundnut oil, castor oil, coconut oil, corn oil, olive oil, palm kernel oil, sunflower oil, soya oil, rapeseed oil, almond oil, grapeseed oil, thistle oil, wheatgerm oil, evening primrose oil, macadamia nut oil, argan oil and/or avocado oil.

Very good results are obtained with linear or branched dialkyl carbonates as a3), preferably with symmetrical linear or branched C₆-C₂₂ dialkyl carbonates.

Examples of symmetrical linear C₆-C₂₂ dialkyl carbonates are di-n-hexyl carbonate, di-n-capryl carbonate, di-n-hexyl carbonate and/or mixtures thereof, especially dicapryl carbonate such as Cetiol® CC, a commercial product from BASF Personal Care & Nutrition GmbH.

Suitable branched C₆-C₂₂ dialkyl carbonates may have the following alkyl radicals: 2-methylpropyl, isobutyl, isopentyl, for example 2,2-dimethylpropyl (=neopentyl), 3-methylbutyl (=isopentyl), isohexyl, isooctyl, for example 2-ethylhexyl or 3-ethylhexyl or 4-ethylhexyl or 5-ethylhexyl radicals, isodecyl radicals, for example trimethylheptyl radicals (=neodecyl radical), isostearyl, isooctyl, isononyl, isodecyl, isotridecyl, 2-ethylbutyl, 2-ethylhexl, 2-propylheptyl, 2-butyloctyl, 2-butyldecyl, 2-hexyloctyl, 2-hexyldecyl, 2-hexcyldodecyl, 2-octyldecyl radical. Symmetrical, branched dialkyl carbonates such as di(2-propyl-1-heptyl) carbonate or di(2-ethyl-1-butyl) carbonate are especially preferred.

Within the linear or branched, symmetrical and/or asymmetrical dialkyl ethers, particular preference is given to the symmetrical dialkyl ethers, especially dialkyl ethers having 6 to 10 carbon atoms per alkyl group, for example a di-n-capryl ether such as Cetiol OE, a commercial product from BASF Personal Care & Nutrition GmbH.

Furthermore, a3) may consist of so-called “CX hydrocarbons” or comprise same, wherein these “CX hydrocarbons” consist only of carbon and hydrogen and have a carbon number of X (wherein X represents an integer). Thus, for example the term C11 hydrocarbon encompasses all hydrocarbons having a carbon number of 11. The term “carbon number” includes all the carbon atoms present in the hydrocarbon. Thus, it is for example equal to 11 for undecane or 13 for tridecane.

According to the present invention, a3) may consist of “CX hydrocarbons” of the hydrocarbon group containing C7, C9, C11, C13, C15, C17, C19, C21 and/or C23 hydrocarbons, or may comprise same. The “CX hydrocarbons” are preferably saturated, and especially saturated and linear, hydrocarbons selected from the group consisting of C7, C9, C11, C13, C15, C17, C19, C21 and/or C23 hydrocarbons. Outstandingly suitable examples are n-heptane, n-nonane, n-undecane, n-tridecane, n-pentadecane, n-heptadecane, n-nonadecane, n-heneicosane and/or n-tricosane and especially n-undecane and/or n-tridecane.

For the purposes of the invention, oils (a3) are preferred that are formed from the group of esters of branched C₆-C₃₂ carboxylic acids with linear C₆-C₂₂ fatty alcohols, linear C₆-C₁₂ dialkyl ethers, symmetrical linear C₆-C₂₂ fatty alcohol dicarbonates, symmetrical branched C₆-C₂₂ dialkyl carbonates and/or saturated and linear “CX” hydrocarbons selected from the group containing C7, C9, C11, C13, C15, C17, C19, C21 and/or C23 hydrocarbons.

Most particularly preferred as oils (a3) are isononanoic esters of cetyl and/or stearyl alcohol, such as Cetiol® SN, a commercial product from BASF Personal Care Nutrition GmbH, dicapryl ethers such as Cetiol® OE, a commercial product from BASF Personal Care & Nutrition GmbH, dicapryl carbonate such as Cetiol® CC, a commercial product from BASF Personal Care & Nutrition GmbH, di(2-propyl-1-heptyl) carbonate such as Cetiol® 4 All, a commercial product from BASF Personal Care & Nutrition GmbH and also n-undecane and/or n-tridecane such as Cetiol® Ulimate, a commercial product from BASF Personal Care & Nutrition GmbH.

Especially preferred as oils (a3) are those that are selected from the group formed of esters of C₆-C₂₂ fatty acids with branched C₆-C₃₂ carboxylic acids, linear C₆-C₁₂ dialkyl ethers and/or symmetrical linear C₆-C₂₂ fatty alcohol dicarbonates or mixtures thereof.

The oils may also comprise solid fats and/or waxes, as long as a3) overall is present as a liquid at room temperature, or can be stirred in or incorporated at room temperature. Typical examples of fats are glycerides, i.e. solid or liquid vegetable or animal products which are composed essentially of mixed glycerol esters of higher fatty acids. Monoglycerides and diglycerides should especially be mentioned here. Possible waxes are, inter alia, natural waxes, for example candelilla wax, carnauba wax, japan wax, esparto grass wax, cork wax, guaruma wax, rice bran oil wax, sugarcane wax, ouricury wax, montan wax, beeswax, shellac wax, spermaceti, lanolin (wool wax), uropygial grease, ceresin, ozokerite (earth wax), petrolatum, paraffin waxes, micro waxes; chemically modified waxes (hard waxes), for example montan ester waxes, Sasol waxes, hydrogenated jojoba waxes, and also synthetic waxes, for example polyalkylene waxes and polyethylene glycol waxes. Tocopherols and essential oils are also suitable as oil components. However, in this context the glyceryl monoesters a2) are not considered to be a constituent of the oil phase a3).

For the purposes of the present teaching, the microemulsion concentrates comprise the oils a3) preferably in quantities of 15 to 35 wt %, preferably in quantities of 15 to 25 wt % - based on microemulsion concentrate.

a4) Amphoteric or Zwitterionic Surfactants

For the purposes of the invention, it is necessary for the amphoteric or zwitterionic surfactants to be selected from the group of amphoacetates. The term amphoacetates is understood to mean the carboxylation products of amidoamines.

Preferred amphoacetates have the formula (II),

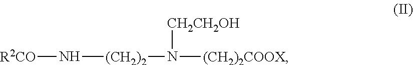

in which R²CO represents an aliphatic acyl radical having 6 to 22 carbon atoms and 0 or 1 to 3 double bonds, and X represents an alkali metal or ammonium.

Typical examples are reaction products of fatty acids having 6 to 22 carbon atoms, namely caproic acid, caprylic acid, capric acid, lauric acid, myristic acid, palmitic acid, palm oleic acid, stearic acid, isostearic acid, oleic acid, elaidic acid, petroselinic acid, linoleic acid, linolenic acid, elaeostearic acid, arachidic acid, gadoleic acid, behenic acid and erucic acid and also technical-grade mixtures thereof, with aminoethylethanolamine (AEEA) and subsequent carboxylation with sodium chloroacetate. Preference is given to using a condensation product of C_{8/18} coconut fatty acid with aminoethylethanolamine (AEEA) and subsequent carboxylation with sodium chloroacetate.

For the purposes of the present invention, the amphoacetates are preferably present in the entire selected quantities of 4.75 to 6.5 wt %—based on the concentrate.

a5) Water

The inventive microemulsion concentrates have a total water content of 0.5 to 35 wt %, preferably 15 to 35 wt % and especially 20 to 25 wt %—based on concentrate.

Here, the total water content includes additionally added water and also the water that is added as part of an aqueous formulation of a further active constituent of the microemulsion, for example of a1), a2) and/or a3).

a6) Polyols and Further Possible Ingredients of the Microemulsion Concentrate

The inventive microemulsion concentrates optionally comprise additional polyols, preferably glycerol, and/or preservatives and/or perfume and/or salts and/or pH regulators.

a6) are preferably present in quantities of 0.5 to 50, and especially in quantities of 25 to 40 wt %—based on microemulsion concentrate.

It is advantageous here if glycerol is present as polyol, and preferably in quantities of 25 to 38 wt %—based on concentrate.

Preservatives are optional and preferably only present in small quantities of 0.05 to 1 wt %, especially in quantities of 0.1 to 0.7 wt %.

Particularly suitable preservatives are benzoic acid and salts thereof, sorbic acid and salts thereof, phenoxyethanol, benzyl alcohol, alkylparabens, preferably ethylparaben, methylparaben and propylparaben.

pH regulators are preferably used if it is desired to adjust the pH to a skin-friendly pH; the pH of the microemulsion concentrates is typically between 4.0 and 5.5. Citric acid is for example suitable for pH adjustment.

As perfume, mixtures of natural and synthetic fragrances are suitable. Natural fragrances are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tertbutylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethol, citronellol, eugenol, isoeugenol, geraniol, linalool, phenylethyl alcohol and terpineol, and the hydrocarbons include primarily the terpenes and balsams. However, preference is given to using mixtures of different fragrances which together produce a pleasant scent note. Essential oils of relatively low volatility, which are mostly used as aroma components, are also suitable as perfume, e.g. sage oil, camomile oil, oil of cloves, melissa oil, mint oil, cinnamon leaf oil, linden blossom oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, Fixolide NP, evernyl, iraldein gamma, phenylacetic acid, geranyl acetate, benzyl acetate, rose oxide, romillat, irotyl and floramat alone or in mixtures.

For the purposes of the invention, very particularly preferably microemulsion concentrates comprise
a1) 5 to 20 wt % of alkyl and/or alkenyl glycosides
a2) 3 to 20 wt % of cosurfactants selected from the group formed of monoesters of glycerol with a C₆-C₂₂ fatty acid
a3) 15 to 35 wt % of oils different to a2)
a4) 4.75 to 6.5 wt % of amphoteric and/or zwitterionic surfactants selected from the group of amphoacetates
a5) 15 to 35 wt % of water, and also
a6) 0.5 to 50 wt % of polyols, preferably glycerol, and/or preservatives and/or perfume and/or salts and/or pH regulators,
   in each case based on concentrate and adding up to 100 wt %.

Especially suitable microemulsion concentrates comprise
a1) 7 to 12 wt % of alkyl and/or alkenyl glycosides
a2) 3 to 7 wt % of cosurfactants selected from the group formed of monoesters of glycerol with a C₆-C₂₂ fatty acid
a3) 15 to 25 wt % of oils different to a2)
a4) 4.75 to 6.5 wt % of amphoteric and/or zwitterionic surfactants selected from the group of amphoacetates
a5) 20 to 30 wt % of water, and also
a6) 25 to 40 wt % of polyols, preferably glycerol, and/or preservatives and/or perfume and/or salts and/or pH regulators,
   in each case based on concentrate and adding up to 100 wt %.

While the inventive microemulsion concentrates may comprise further constituents than a1) to a6), for the purposes of the invention it is however most particularly preferable if the inventive microemulsions only consist of the described constituents a1) to a6) in the preferred stated quantities. Microemulsion concentrates that are free of compounds comprising ethylene oxide or other alkoxylation products are especially preferred.

Method for Preparing the Microemulsion Concentrates

A further subject of the present invention is a method for preparing microemulsion concentrates according to claim 1, wherein a mixture is prepared of a1) alkyl and/or alkenyl glycosides, a4) amphoteric or zwitterionic surfactants selected from the group of amphoacetates in the presence of a5) water and optionally a6) polyols and/or preservatives and/or perfume and/or salts and/or pH regulators, this is heated and, simultaneously or in succession, the a3) oils different to a2) and the cosurfactant a2) selected from the group formed of monoesters of glycerol with a C₆-C₂₂ fatty acid are added thereto.

Accordingly, in a preferred embodiment a mixture (I) is prepared from alkyl and/or alkenyl glycosides a1) and amphoteric or zwitterionic surfactants a4) and water a5) and polyols, especially glycerol, as a6). In this mixture (I), the further ingredients from a6) such as preservatives and/or pH regulators and/or perfume and/or salts may also be present. The water in the mixture (I) is generally a result of the aqueous formulations of the alkyl and/or alkenyl glycosides a1) and amphoteric or zwitterionic surfactants a4). The mixture (I) is then preferably heated to approximately 50 to 80° C., especially 65 to 75° C., with stirring. As soon as the mixture (I) is clearly dissolved, the oil a3) and then the cosurfactant a2) are preferably added thereto in succession, and preferably further stirred at approximately 70 to 80° C. A further stirring time of approximately 10 to 30 minutes is recommended. After cooling, the pH may be adjusted.

Clear, storage-stable microemulsion concentrates are obtained. This is all the more surprising since this does not occur with betaines.

A further subject of the present invention is therefore the use of a mixture of a1) alkyl and/or alkenyl glycosides and a4) amphoteric and/or zwitterionic surfactants selected from the group of amphoacetates as emulsifier or surfactant mixture for preparing stable microemulsion concentrates according to claim 1 having a total water content of 0.5 to 35 wt %.

For the purposes of the invention, it has proved advantageous when the quantitative ratio of a1):a4) is in the range from 1.9:1 to 1.45:1—in the concentrate.

For the purposes of the invention, specifically the use of the combination of a1) and a4) is advantageous for the very good shelf life of the inventive microemulsion concentrates.

The inventive microemulsion concentrates may be converted into any desired aqueous dilutions with water, without an undesired gelling or disruption of the emulsion occurring during the dilution.

Microemulsion Concentrates and Use Thereof

Since the aqueous dilutions of the microemulsion concentrates have outstanding wettability of paper and textile, a further subject of the present invention relates to the use of the inventive microemulsion concentrates according to one of claims 1 to 10 in the form of the aqueous dilution thereof, as agent for impregnating paper and textile for the preparation of sheet-like articles in cosmetics, especially what are referred to as wet wipes.

The inventive microemulsion concentrates are preferably present in quantities of 0.1 to 10 wt % based on the aqueous dilution or impregnating agent.

The umbrella term “textile and paper” is understood to mean various different varieties and articles which may sometimes differ considerably in their fields of application and their nature. For example, tissue papers and/or tissue cloth and/or tissues (referred to below as tissues) are suitable. These may be single- or multi-layered. Generally, the papers have a weight per square meter of 10 to 65, preferably 15 to 30 g, and a density of 0.6 g/cm and less. Examples of tissue papers are toilet papers, pocket paper tissues, facial cleansing wipes, make-up removal wipes, refreshing wipes, household wipes and the like. In addition to the paper-based tissues, corresponding tissue cloths are also suitable which are made from fibrous material or fleece material.

Preference is given to multi-layered tissues. Preference is especially given to those tissues which have an impermeable and/or semi-permeable barrier layer between the individual layers. The semi-permeable barrier layer can, for example, be designed as a semi-permeable membrane. With such wipes, two or more compositions (optionally after prior dilution) can be applied to a cloth. This may be very particularly preferred in order to effect cleaning with one side of the wipes by means of the composition applied to the cloth. The other side can then be used for rubbing, for example, for drying or optionally for applying a care active ingredient to the skin.

Furthermore, the wipes may consist of at least 3 layers of tissue treated with compositions (optionally after prior dilution). Advantageously, then, one layer of cloth is formed as a semipermeable membrane between at least two layers of treated cloth in each case. The semipermeable membrane is therefore permeable in the direction of the outer cloth layers. Thus, for example, on the inside, a composition (optionally after prior dilution) may be applied to the innermost layer, which is either immiscible and/or not stable with respect to the composition applied to the outer side. This makes it possible to offer “two-in-one cloths” for cleansing and care.

The different coloring of the cloth layers and also the different construction of the cloths from two or more materials, especially with respect to the absorbency and permeability of the different cloth layers, is also possible.

Furthermore, textile fibers, for example, are suitable both from natural fibers, such as cellulose, silk, wool, regenerated cellulose (viscose, rayon), cellulose derivatives, and textile fibers from synthetic fibers such as polyester, polypropylene, polyethylene terephthalate, polyamide, polyolefin and polyacrylonitrile fibers or mixtures of such fibers. These fibers may be woven or nonwoven.

The term “impregnating agent” in this case encompasses an agent for impregnation, with “impregnation” intended to mean any type of application of a liquid to one or more solid surfaces of the above-described type, for example soaking, coating, sprinkling or spraying, dipping, finishing, stripping, etc.

The inventive microemulsion concentrates are preferably used as impregnating agent in the form of an aqueous dilution, wherein further additional cosmetic active ingredients and/or auxiliaries may be present, depending on the desired application. These may be the same cosmetic active ingredients and/or auxiliaries which have already been described under a6) in connection with the inventive microemulsion concentrates or else further customary active ingredients and auxiliaries known to those skilled in the art which are applied in aqueous dilutions to textile and paper and, as sheet-like articles in cosmetics, are marketed especially as so-called wet wipes.

Surfactants are typically present in wet wipes formulations for cleaning the skin.

A further subject of the present invention is therefore agents for impregnating paper and textile for the preparation of sheet-like articles in cosmetics, comprising
A) microemulsion concentrates according to claim 1, and
B) water, and also optionally
C) further cosmetic ingredients selected from the group of anionic/nonionic, cationic and/or zwitterionic surfactants, and also optionally
D) further ingredients different to C).

The anionic surfactants are preferably selected from the group formed of soaps, alkylbenzenesulfonates, alkanesulfonates, olefinsulfonates, α-methyl ester sulfonates, sulfo fatty acids, alkyl sulfates, alkyl ether sulfates, hydroxy mixed ether sulfates, monoglyceride sulfates, fatty acid amide sulfates, mono- and dialkyl sulfosuccinates, mono- and dialkyl sulfosuccinamates, sulfotriglycerides, amide soaps, fatty acid isethionates, fatty acid sarcosinates, fatty acid taurides, N-acylamino acids, for example acyl lactylates, acyl tartrates, acyl glutamates and acyl aspartates, alkyl oligoglucoside sulfates, alkyl oligoglucoside carboxylates, protein fatty acid condensates (especially wheat-based plant products) and alkyl phosphates; particular preference is given to alkyl sulfates alone or in a mixture with further mild anionic surfactants such as acyl glutamates and/or alkyl oligoglucoside carboxylates.

Alkyl sulfates (“fatty alcohol sulfates”) represent known anionic surfactants which are prepared on an industrial scale by SO₃ or chlorosulfonic acid (CSA) sulfation of fatty alcohol and subsequent neutralization. For the purposes of the invention, sulfates are considered that have the formula

R⁵O—SO₃X′

in which R⁵ represents a linear or branched alkyl and/or alkenyl radical having 6 to 22 carbon atoms and X′ represents an alkali and/or alkaline earth metal, ammonium, alkylammonium, alkanolammonium or glucammonium. Typical examples are the sulfates of caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachidyl alcohol, gadoleyl alcohol, behenyl alcohol, erucyl alcohol and brassidyl alcohol and also technical-grade mixtures thereof in the form of their sodium and/or magnesium salts.

Particular preference is given to the sodium salt of the sulfate based on the alcohol derived from coconut fatty acid.

In terms of nonionic surfactants, preference is given to fatty alcohol polyglycol ethers, alkylphenol polyglycol ethers, fatty acid polyglycol ethers, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (especially wheat-based plant products), polyol fatty acid esters, sugar esters, sorbitan esters, polysorbates and amine oxides. If the nonionic surfactants contain polyglycol ether chains, these may have a conventional homolog distribution, but preferably have a narrow homolog distribution. Preference is given to nonionic surfactants without ethylene oxide and/or propylene oxide units.

Zwitterionic surfactants refer to those surface-active compounds which bear at least one quaternary ammonium group and at least one —COO(—)— or —SO3(—)— group in the molecule. Particularly suitable zwitterionic surfactants are the so-called betaines, such as the N-alkyl-N,Ndimethylammonium glycinates, for example cocoalkyldimethylammonium glycinate, Nacylaminopropyl-N,N-dimethylammonium glycinates, for example cocoacylaminopropyldimethylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic surfactant is the fatty acid amide derivative known under the INCI name Cocamidopropyl Betaine.

Typical examples of amphoteric or zwitterionic surfactants are alkyl betaines, alkylamido betaInes, aminopropionates, aminoglycinates, imidazolinium betaines and sulfo betaines. The surfactants mentioned are exclusively known compounds. With regard to the structure and preparation of these substances, reference may be made to relevant review works in this field. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ethercarboxylic acids, alkyl oligoglucosides and/or mixtures thereof with alkyl oligoglucoside carboxylates, fatty acid glucamides, alkylamidobetaines, amphoacetates and/or protein fatty acid condensates, the latter preferably based on wheat proteins or salts thereof.

Usable cationic surfactants are especially quaternary ammonium compounds. Preference is given to ammonium halides, especially chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. In addition, the very readily biodegradable quaternary ester compounds, for example the dialkylammonium methosulfates and methylhydroxyalkyldialkoyloxyalkylammonium methosulfates sold under the trade name Stepantex° and the corresponding products of the Dehyquart® series can be used as cationic surfactants. The term “ester quats” is generally understood to mean quaternized fatty acid triethanolamine ester salts. They can impart an exceptional soft feel to the inventive preparations. These are known substances which are prepared by the relevant methods of organic chemistry. Further cationic surfactants usable in accordance with the invention are the quaternized protein hydrolyzates.

For the purposes of the invention, surfactants having alkylene oxide units may be present, but preferably exclusively surfactants without alkylene oxide units are present.

The inventive impregnating agents may also comprise, if desired depending on the application, further ingredients D) that are known to those skilled in the art, for example emulsifiers, pearlizing waxes, stabilizers, salt, thickeners, consistency regulators, self-tanning agents, pigments, antioxidants, antidandruff agents, film-formers, swelling agents, insect repellants, deodorants and antiperspirants, biogenic active ingredients, humectants (e.g. glycerol, sorbitol) and/or further pH regulators. As biogenic active ingredients, preference is given especially to tocopherol, tocopherol acetate, tocopherol palmitate, deoxyribonucleic acid, coenzyme Q10, ascorbic acid, retinol and retinyl derivatives, bisabolol, allantoin, phytantriol, panthenol, AHA acids, amino acids, ceramides, essential oils, hyaluronic acid, creatine, protein hydrolyzates, plant extracts, peptides and vitamin complexes. Suitable consistency regulators are primarily fatty alcohols or hydroxy fatty alcohols having 12 to 22 and preferably 16 to 18 carbon atoms and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates.

Suitable thickeners are for example aerosil types (hydrophilic silicas), polysaccharides, especially xanthan gum, guar gum, agar agar, alginates and tyloses, carboxymethylcellulose, hydroxyethylcellulose and hydroxypropylcellulose, further higher molecular-weight polyethylene glycol monoesters and diesters of fatty acids, polyacrylates and hydrophobically modified polyacrylates, polyacrylamides, polymers, polyvinyl alcohol and polyvinylpyrrolidone. Bentonites which are a mixture of cyclopentasiloxane, disteardimonium hectorite and propylene carbonate have proved to be particularly effective. Additionally possible are esters of fatty acids with polyols, for example pentaerythritol or trimethylolpropane, such as Arlypon® TT, a commercial product from BASF Personal Care and Nutrition GmbH, and also electrolytes such as sodium chloride and ammonium chloride. Mixtures of xanthan gum are particularly preferred as thickener.

If more preservatives are used, these are preferably selected from the group already mentioned in connection with the microemulsion concentrates, for example benzoic acid and salts thereof, citric acid and salts thereof, phenoxyethanol, benzyl alcohol, alkyl parabens, preferably ethylparaben, methylparaben and propylparaben, or formaldehyde solution, pentanediol or sorbic acid and also the silver complexes known under the name Surfacine®, and the additional substance classes listed in Annex 6, parts A and B, of the Cosmetics Directive.

If stronger perfuming is desired, further perfumes may of course be present in the impregnating agent, for example the perfumes of the microemulsion concentrates already listed under a6).

Furthermore, water-soluble thickeners, such as natural and/or synthetic polymers such as xanthan gum, hydroxyethylcellulose, polyvinylpyrrolidone or high molecular weight polyethylene oxides, are often also present as auxiliaries.

Hydrotropes may also be present to improve the flow behavior. Typical examples are glycerol; alkylene glycols such as ethylene glycol, diethylene glycol, propylene glycol, butylene glycol, hexylene glycol and polyethylene glycols having an average molecular weight of 100 to 1000 Dalton; technical-grade oligoglycerol mixtures having a degree of self-condensation of 1.5 to 10 such as technical-grade diglycerol mixtures having a diglycerol content of 40 to 50% by weight; methylol compounds such as, especially, trimethylolethane, trimethylolpropane, trimethylolbutane, pentaerythritol and dipentaerythritol; lower alkyl glucosides, especially those having 1 to 8 carbon atoms in the alkyl radical, for example methyl glucoside and butyl glucoside; sugar alcohols having 5 to 12 carbon atoms, for example sorbitol or mannitol, sugars having 5 to 12 carbon atoms, for example glucose or sucrose; amino sugars, for example glucamine and/or dialcoholamines such as diethanolamine or 2-amino-1,3-propanediol. If glycerol is present as hydrotrope, this is added in addition to the stated quantity of polyol in the inventive concentrate.

Suitable cationic polymers are, for example, cationic cellulose derivatives, such as a quaternized hydroxyethylcellulose obtainable under the name Polymer JR 400® from Amerchol, cationic starch, copolymers of diallylammonium salts and acrylamides, quaternized vinylpyrrolidone/vinylimidazole polymers, such as Luviquat® (BASF), condensation products of polyglycols and amines, quaternized collagen polypeptides, for example lauryldimonium hydroxypropyl hydrolyzed collagen (Lamequat® L/Grünau), quaternized wheat polypeptides, polyethyleneimine, cationic silicone polymers such as amodimethicones, copolymers of adipic acid and dimethylaminohydroxypropyldiethylenetriamine (Cartaretine®/Sandoz), copolymers of acrylic acid with dimethyldiallylammonium chloride (Merquat® 550/Chemviron), polyaminopolyamides such as described in FR 2252840 A for example and also cross-linked water-soluble polymers thereof, cationic chitin derivatives such as quaternized chitosan, optionally in microcrystalline distribution, condensation products of dihaloalkylene, for example dibromobutane with bisdialkylamines, such as bisdimethylamino-1,3-propane, cationic guar gum such as Jaguar® CBS, Jaguar® C-17, Jaguar® C-16 from Celanese, quaternized ammonium salt polymers such as Mirapol® A-15, Mirapol® AD-1, Mirapol® AZ-1 from Miranol.

Useful anionic, zwitterionic, amphoteric and non-ionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, non-crosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers and optionally derivatized cellulose ethers and silicones. Further suitable polymers and thickeners are listed in Cosm.Toil. 108, 95 (1993).

Suitable silicone compounds are, for example, dimethylpolysiloxanes, methylphenylpolysiloxanes, cyclic silicones, and amino-, fatty acid-, alcohol-, polyether-, epoxy-, fluorine-, glycosideand/or alkyl-modified silicone compounds, which at room temperature may either be liquid or else in the form of a resin. Also suitable are simethicones, which are mixtures of dimethicones with an average chain length of from 200 to 300 dimethylsiloxane units and hydrogenated silicates. A detailed overview of suitable volatile silicones is, furthermore, given by Todd et al. in Cosm. Toil. 91, 27 (1976).

Biogenic active ingredients are understood to mean, for example, tocopherol, tocopherol acetate, tocopherol palmitate, ascorbic acid, (deoxy)ribonucleic acid and fragmentation products thereof, retinol, bisabolol, allantoin, phytantriol, panthenol, AHA acids, amino acids, ceramides, pseudo ceramides, essential oils, plant extracts and vitamin complexes.

Suitable insect repellents are N,N-diethyl-m-toluamide, 1,2-pentanediol or ethyl butylacetylaminopropionate.

Suitable UV light protection filters are organic substances that are liquid at room temperature or crystalline (light protection filters) which are capable of absorbing ultraviolet rays and releasing the energy absorbed again in the form of longer-wave radiation, for example heat. UV filters may be oil-soluble or water-soluble. Examples of typical oil-soluble UV-B filters or broadspectrum UV-NB filters include:

3-benzylidenecamphor or 3-benzylidenenorcamphor (Mexoryl SDS 20) and derivatives thereof, e.g. 3-(4-methylbenzylidene)camphor, as described in EP 0693471 B1

3-(4′-trimethylammonium)benzylidenebornan-2-one methylsulfate (Mexoryl SO)

3,3′-(1,4-phenylenedimethine)bis(7,7-dimethyl-2-oxobicyclo[2.2.1]heptane-1-methanesulfonic acid) and salts (Mexoryl SX)

3-(4′-sulfo)benzylidenebornan-2-one and salts (Mexoryl SL)

polymer of N-[2(and 4)-(2-oxoborn-3-ylidenemethyl)benzyl]acrylamide (Mexoryl SW)

2-(2H-benzotriazol-2-yl)-4-methyl-6-(2-methyl-3-(1,3,3,3-tetramethyl-1-(trimethylsilyloxy)disiloxanyl)propyl)phenol (Mexoryl XL)

4-aminobenzoic acid derivatives, preferably 2-ethylhexyl 4-(dimethylamino)benzoate, 2-octyl 4(dimethylamino)benzoate and amyl 4-(dimethylamino)benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4methoxy-4′-methylbenzophenone, 2,2′-dihydroxy-4-methoxybenzophenone;

esters of benzalmalonic acid, preferably di-2-ethylhexyl 4-methoxybenzmalonate;

triazine derivatives, for example 2,4,6-trianilino(p-carbo-2′-ethyl-1′-hexyloxy)-1,3,5-triazine and

2,4,6-tris[p-(2-ethylhexyloxycarbonyl)anilino]-1,3,5-triazine (Uvinul T 150), as described in EP 0818450 A1, or bis(2-ethylhexyl) 4,4′-[(6-[4-(1,1-dimethylethy)aminocarbonyl)phenylamino]-1,3,5-triazine-2,4-diyl)diimino]benzoate (Uvasorb® HEB);

2,2-(methylenebis(6-(2H-benzotriazol-2-yl)-4-(1,1,3,3-tetramethylbutyl)phenol) (Tinosorb M);

2,4-bis[4-(2-ethylhexyloxy)-2-hydroxyphenyl]-6-(4-methoxyphenyl)-1,3,5-triazine (Tinosorb S);

propane-1,3-diones, for example 1-(4-tert-butylphenyl)-3-(4′-methoxyphenyl)propane-1,3-dione;

ketotricyclo(5.2.1.0)decane derivatives, as described in EP 0694521 B1;

dimethicodiethyl benzalmalonates (Parsol SLX).

Useful water-soluble UV filters include:

2-phenylbenzimidazole-5-sulfonic acid and the alkali metal, alkaline earth metal, ammonium, alkylammonium, alkanolammonium and glucammonium salts thereof;

2,2-(1,4-phenylene)bis(1H-benzimidazole-4,6-disulfonic acid, monosodium salt) (Neo Heliopan AP)

sulfonic acid derivatives of benzophenones, preferably 2-hydroxy-4-methoxybenzophenone-5sulfonic acid and salts thereof;

sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful typical UV-A filters are especially derivatives of benzoylmethane, for example 1-(4′-tertbutylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF), and also hexyl 2-[4-(diethylamino)-2hydroxybenzoyl]benzoate (Uvinul® A plus).

The UV-A and UV-B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters, for example 2-phenylbenzimidazole-5-sulfonic acid and the alkali metal, alkaline earth metal, ammonium, alkylammonium, alkanolammonium and glucammonium salts thereof.

Suitable UV light protection filters are especially the substances approved according to Annex VII of the Commission Directive (in the version: Commission Directive 2005/9/EC of Jan. 28, 2005 amending Council Directive 76/768/EEC, concerning cosmetic products, for the purposes of adapting Annexes VII thereof to technical progress), to which reference is explicitly made here.

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be present in surface-treated form, i.e. hydrophilized or hydrophobized. Typical examples are coated titanium dioxides, for example T 805 titanium dioxide (Degussa) or Eusolex® T, Eusolex® T-2000, Eusolex® T-Aqua, Eusolex® AVO, Eusolex® T-ECO, Eusolex® T-OLEO and Eusolex® T-S (Merck).

Typical examples of zinc oxides are, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coatings are in particular silicones and specifically trialkoxyoctylsilanes or simethicones. In sunscreen compositions, preference is given to using micropigments or nanopigments. Preference is given to using micronized zinc oxide. Further suitable UV light protection filters are given in the overview by P. Finkel in SOFW-Joumal 122, 8/1996, pp. 543-548 and Parf. Kosm. 80. No. 3/1999, pp. 10 to 16.

In addition to the two aforementioned groups of primary light protection substances, it is also possible to use secondary light protection agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,Lcarnosine, D-carnosine, L-carnosine and derivatives thereof (e.g. anserine), carotenoids, carotenes (e.g. alpha-carotene, beta-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. α-hydroxy fatty acids, palmitic acid, phytic acid, lactoferrin), α-hydroxy acids (e.g. citric acid, lactic acid, malic acid), humic acid, gallic acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

Typical examples of solubilizers are INCI: Polysorbate 20 (Eumulgin® SML 20), INCI: Polysorbate 80 (Eumulgin® SMO 20), INCI: Polysorbate 60 (Eumulgin® SMS 20), hydrogenated castor oil with different degrees of ethoxylation, for example with 40 EO (Eumulgin® CO 40).

The inventive impregnating agents preferably comprise
A) 0.1 to 10 wt % of microemulsion concentrates and
B) 90 to 99.8 wt % of water
C) 0.05 to 10 wt % of C) and
D) 0.05% to 10 wt % of D).

The inventive impregnating agents are storage stable across broad pH ranges and are preferably adjusted to pHs of 4.0 to 7.5.

The inventive impregnating agents are optically homogeneous, that is to say that they are perceived as a single phase by the naked eye. Furthermore, they exhibit good shelf lives and low viscosity, even on long-term storage; preferably the values are less than 400, especially less than 200 mPas.

The sheet-like articles made of textiles or papers impregnated in this way are preferably used in cosmetics in the field of so-called wet wipes for skincare or skin cleaning (especially for baby care or cleaning). Examples which may be mentioned are care or cleansing wipes for facial skin (so-called facial tissues, make-up removal wipes etc.), refreshing wipes for skin, antibacterial and/or deodorizing wipes, products for intimate care, for example tampons, sanitary towels, panty liners, intimate wipes), moist toilet paper, incontinence products, self-tanning cloths or so-called insect repellent cloths.

### EXAMPLES

The microemulsion concentrates were prepared as follows:

Glycerol, Plantacare® 1200UP, benzoic acid, approximately 50-60% of the total quantity of citric acid solution, and Dehyton® MC were added to a vessel heated to 45° C. and stirred at a moderate stirring speed, then heated to 70° C. Once everything was clearly dissolved, Cetiol® SN and Monomuls® 90-018 were added and stirred at 75° C. for 15 minutes to 30 minutes. The mixture was left to cool to 50° C. and the remaining quantity of citric acid solution was added.

Further cooling to approximately 30° C. and testing of pH; optionally further dosing with citric acid solution to adjust to the desired pH, approximately pH 4.5.

Compounds used:

Plantacare® 1200UP (INCI: Laurylglucoside); alkypolyglycoside a1)

Cetiol®SN (INCI: Cetearyl Isononanoate); oil a3)

Dehyton® MC (INCI: Sodium Cocoamphoacetate); amphoteric or zwitterionic surfactant a4)

Monomuls® 90-018 (INCI: Glyceryl Oleate) cosurfactant a2)

Citric acid solution (50% by weight).

The compounds were used in the following quantities:

### A) Microemulsion Concentrates Example 1a-1e with Different Oils

| | | wt % | wt % | wt % | wt % | wt % | wt % |
|---|---|---|---|---|---|---|---|
| | | (raw | (active | (active | (active | (active | (active |
| | | material) | matter) | matter) | matter) | matter) | matter) |
| Product | INCI | 1a | 1a | 1b | 1c | 1d | 1e |
| Cetiol ® SN | Cetearyl Isononanoate | 17.00 | 17.00 | | | | |
| Cetiol ® CC | Di-n-Octylcarbonate | | | 21 | | | |
| Cetiol ® OE | Di-n-Octylether | | | | 21 | | |
| Cetiol ® 4 All | Dipropylheptylcarbonate | | | | | 17 | |
| Cetiol ® Ultimate | Undecane/Tridecane | | | | | | 25 |
| Monomuls ® 90- | Glyceryl Oleate | 4.20 | 4.20 | 3.70 | 3.70 | 4.20 | 3.00 |
| O18 | | | | | | | |
| Dehyton ® MC | Sodium Cocoamphoacetate | 17.80 | 5.87 | 5.87 | 5.87 | 5.87 | 5.87 |
| Plantacare ® 1200 | Lauryl Glucoside | 18.90 | 9.45 | 9.45 | 9.45 | 9.45 | 9.45 |
| UP | | | | | | | |
| Glycerol | Glycerol | 37.92 | 37.92 | 34.42 | 34.42 | 37.92 | 31.12 |
| Benzoic Acid | Benzoic Acid | 0.50 | 0.50 | 0.50 | 0.50 | 0.50 | 0.50 |
| Citric Acid Sol. | Citric Acid | 3.68 | 1.84 | 1.84 | 1.84 | 1.84 | 1.84 |
| (50%) | | | | | | | |
| Sodium Chloride | Sodium Chloride | 0.00 | 1.25 | 1.25 | 1.25 | 1.25 | 1.25 |
| Water demin. | | 0.00 | 21.97 | 21.97 | 21.97 | 21.97 | 21.97 |
| sum | | 100.00 | 100.00 | 100 | 100 | 100 | 100 |

Concentrates with excellent shelf life were formed. After 4 weeks' storage at −5° C., 5° C., room temperature and 40° C., the concentrates were still clear and the viscosity was unchanged. In all concentrates, the transparency before and after storage was measured as FNU less than 10. The viscosity after storage was also <350 m Pas (measurement conditions: 23° C., spindle 1, 20 rpm).

Example 1a) was stored for 12 weeks at the different temperatures; the concentrate 1 a) was also clear after the 12-week storage and the viscosity was unchanged.

Microemulsion concentrates with amphoacetate in different wt % (active matter): Inventive examples 2-4 and comparative examples 1+2

| | | Comparative | | | | Comparative |
|---|---|---|---|---|---|---|
| | | example 1 | Example 2 | Example 3 | Example 4 | example 2 |
| | INCI | [%] | [%] | [%] | [%] | [%] |
| Cetiol ® SN | Cetearyl | 21.00 | 21.00 | 21.00 | 21.00 | 21.00 |
| | Isononanoate | | | | | |
| Plantacare ® | Lauryl Glucoside | 9.45 | 9.45 | 9.45 | 9.45 | 9.45 |
| 1200UP | | | | | | |
| Dehyton ® | Sodium Cocoamphoacetate | 4.70 | 4.99 | 5.87 | 6.46 | 6.76 |
| MC | | | | | | |
| Monomuls ® | Glyceryl Oleate | 5.10 | 5.10 | 5.10 | 5.10 | 5.10 |
| 90-O18 | | | | | | |
| Glycerol | Glycerine | 36.58 | 35.69 | 33.02 | 31.24 | 30.35 |
| Benzoic | Benzoic Acid | 0.50 | 0.50 | 0.50 | 0.50 | 0.50 |
| acid | | | | | | |
| Water | Aqua | 19.83 | 20.37 | 21.97 | 23.04 | 23.57 |
| dem. | | | | | | |
| Sodium | | 1.00 | 1.06 | 1.25 | 1.37 | 1.43 |
| Chloride | | | | | | |
| Citric acid | Citric Acid | 1.84 | 1.84 | 1.84 | 1.84 | 1.84 |
| solution | | | | | | |
| Appearance | | cloudy; | clear; | clear; | clear; microemulsion | cloudy; |
| of the | | two- | microemulsion | microemulsion | | two- |
| concentrate | | phase | | | | phase |

The microemulsions according to the inventive examples 2, 3 and 4 were clear and the viscosity of the microemulsions was also unchanged after 4 weeks' storage: <350 m Pas; (measurement conditions: 23° C., spindle 1, 20 rpm).

Microemulsion with other surfactant: Comparative example 3 with betaine as zwitterionic surfactant

| | INCI | [%] |
|---|---|---|
| Cetiol ® SN | Cetearyl Isononanoate | 21.00 |
| Plantacare ® 1200UP | Lauryl Glucoside | 9.45 |
| Dehyton ® PK45 | cocamidopropylbetaine | 5.87 |
| Monomuls ® 90-O18 | Glyceryl Oleate | 5.10 |
| Glycerol | Glycerine | 34.94 |
| Benzoic acid | Benzoic Acid | 0.50 |
| Water dem. | Aqua | 21.29 |
| Citric acid solution | Citric Acid | 1.84 |
| Appearance of the concentrate | | creamy emulsion |

wt % (active matter)

B) Use Examples: Impregnating Agents

Impregnating agents were prepared comprising the microemulsion according to example 1a). The % are given as percentage by weight of raw material.

### Example 5

5.0% microemulsion according to example 1a)

0.5% sodium benzoate

0.1% Cotton Touch® perfume from Symrise

0.1% Eumulgin® CO 40 as solubilizer (=hydrogenated castor oil +40 EO)

q.s. Citric Acid (50% strength) for pH adjustment to pH 4.8-5.1 up to 100% water, demin.

### Example 6

5.0% microemulsion according to example 1a)

0.6% Rheocare® HSP 1180 (Polyacrylamidomethylpropane Sulfonic Acid)

0.1% Cotton Touch perfume from Symrise

1.0% Phenoxyethanol

q.s. KOH (20% strength) for pH adjustment to pH 6.0-6.5 up to 100% water, demin.

### Example 7

5.0% microemulsion according to example 1a)

0.5% sodium benzoate

0.1% Cotton Touch perfume from Symrise

0.1% Xanthan gum

q.s. Citric Acid (50% strength) for pH adjustment to pH 4.8-5.1 up to 100% water, demin.

### Example 8

3.3% microemulsion according to example 1a)

0.1% Cotton Touch® perfume from Symrise

0.5% sodium benzoate

q.s. Citric Acid (50% strength) for pH adjustment to pH 4.8-5.1 up to 100% water, demin.

### Example 9

3.3% microemulsion according to example 1a)

0.1% Cotton Touch® perfume from Symrise

1.0% Phenoxyethanol

q.s. NaOH solution (10% strength) for pH adjustment to pH 6.0-7.0 up to 100% water, demin.

### Example 10

3.3% microemulsion according to example 1a)

0.1% Cotton Touch® perfume from Symrise

1.0% Euxyl® K700 from Schülke (Phenoxyethanol, Benzyl Alcohol, Potassium Sorbate, Tocopherol)

q.s. Citric Acid (50% strength) for pH adjustment to pH 4.8-5.1 up to 100% water, demin.

### Example 11

5.0% microemulsion according to example 1a)

0.5% sodium benzoate

0.1% Cotton Touch perfume from Symrise

0.4% D-Panthenol 75W

q.s. Citric Acid (50% strength) for pH adjustment to pH 4.8-5.1 up to 100% water, demin.

### Example 12

5.0% microemulsion according to example 1a)

0.5% sodium benzoate

0.1% Cotton Touch perfume from Symrise

0.3% Aloe Vera Gel Concentrate 10/1 from Symrise

q.s. Citric Acid (50% strength) for pH adjustment to pH 4.8-5.1 up to 100% water, demin.

According to examples 5 to 12, in each case optically homogeneous, clear to cloudy impregnating agents were formed, which were also preserved after 4 weeks' storage at room temperature. Furthermore, the viscosity after 4 weeks' storage also remained satisfyingly low (max. 100 mPas measured at RT), and so the impregnating agents also have a good shelf life.

1. Microemulsion concentrates for impregnating textile and paper in cosmetics, comprising
a1) an alkyl and/or alkenyl glycoside,
a2) a cosurfactant selected from a monoester of glycerol with a C₆-C₂₂ fatty acid,
a3) a an oil other than a2),
a4) an amphoteric or zwitterionic surfactant selected from an amphoacetate in quantities of 4.75 to 6.5 wt %, based on concentrate, and
a5) water,
wherein the total water content is 0.5 to 35 wt %, based on concentrate.

2. The microemulsion concentrates according to claim 1, comprising
a1) an alkyl and/or alkenyl glycoside of formula (I)
R¹O-[G]ₚ (I)
in which R¹ represents an alkyl and/or alkenyl radical having 4 to 22 carbon atoms,
G represents a sugar radical having 5 or 6 carbon atoms, and p represents numbers from 1 to 10.

3. The microemulsion concentrates according to claim 1, comprising a2) a monoester of glycerol formed of monoesters having a C₆-C₂₂ fatty acid mixture comprising unsaturated C₁₂-C₂₂ fatty acids.

4. The microemulsion concentrates according to claim 1, comprising a3) an oil formed from the group of esters of unbranched C₆-C₃₂ carboxylic acids with linear fatty alcohols C₆-C₂₂, linear C₆-C₁₂ dialkyl ethers, symmetrical linear C₆-C₂₂ fatty alcohol dicarbonates, symmetrical branched C₆-C₂₂ dialkyl carbonates, and/or saturated and linear “CX” hydrocarbons selected from the group containing C7, C9, C11, C13, C15, C17, C19, C21 and/or C23 hydrocarbons.

5. The microemulsion concentrates according to claim 1, comprising a3) an oil selected from formed of esters of C₆-C₂₂ fatty acids with branched C₆-C₃₂ carboxylic acids, linear C₆-C₁₂ dialkyl ethers, linear symmetrical C₆-C₂₂ fatty alcohol dicarbonates, or mixtures thereof.

6. The microemulsion concentrates according to claim 1, comprising, as
a4) an amphoacetate of formula (II), 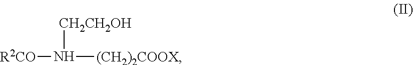
in which R²CO represents an aliphatic acyl radical having 6 to 22 carbon atoms and 0 or 1 to 3 double bonds, and X represents an alkali metal or ammonium.

7. The microemulsion concentrates according to claim 1, additionally comprising a6) polyols, and/or preservatives and/or perfume and/or salts and/or pH regulators.

8. The microemulsion concentrates according to claim 1, wherein the total water content a5) is 20 to 25 wt %, based on concentrate.

9. The microemulsion concentrates according to claim 1, comprising
a1) 5 to 20 wt % of alkyl and/or alkenyl glycoside,
a2) 3 to 20 wt % of the cosurfactant selected from a monoester of glycerol with a C₆-C₂₂ fatty acid,
a3) 15 to 35 wt % of oils different to a2),
a4) 4.75 to 6.5 wt % of the amphoteric and/or zwitterionic surfactant selected from amphoacetates,
a5) 15 to 35 wt % of water, and
a6) 0.5 to 50 wt % of the polyol, and/or preservatives and/or perfume and/or salts and/or pH regulators,
in each case based on concentrate and adding up to 100 wt %.

10. The microemulsion concentrates according to claim 9, comprising
a1) 7 to 12 wt % of the alkyl and/or alkenyl glycoside,
a2) 3 to 7 wt % of the cosurfactant formed of a monoester of glycerol with a 0₆⁻0₂₂ fatty acid,
a3) 15 to 25 wt % of the oil different to a2),
a4) 4.75 to 6.5 wt % of the amphoteric and/or zwitterionic surfactant selected from the group of amphoacetates,
a5) 20 to 30 wt % of water, and
a6) 25 to 40 wt % of the polyol, and/or preservatives and/or perfume and/or salts and/or pH regulators,
in each case based on concentrate and adding up to 100 wt %.

11. A mixture of a1) an alkyl and/or alkenyl glycoside and a4) an amphoteric and/or zwitterionic surfactant selected from amphoacetates for use as an emulsifier or surfactant mixture for preparing stable microemulsion concentrates having a total water content of 0.5 to 35 wt %.

12. A method for preparing microemulsion concentrates according to claim 1, wherein a mixture is prepared of a1) an alkyl and/or alkenyl glycoside, a4) an amphoteric or zwitterionic surfactant from the group of amphoacetates in the presence of a5) water, and optionally a6) polyols and/or preservatives and/or perfume and/or salts and/or pH regulators, is heated and, simultaneously or in succession, the a3) oil different to a2) and the cosurfactant a2) formed of a monoester of glycerol with a C₆⁻0₂₂ fatty acid are added thereto.

13. The use of the microemulsion concentrates according to claim 1, in the form of the aqueous dilution thereof, for use as an agent for impregnating paper and textile for the preparation of sheet-like articles in cosmetics.

14. The microemulsion concentrates for use according to claim 13, wherein the concentrate is used in quantities of 0.1 to 10 wt %, based on aqueous dilution.

15. An agent for impregnating paper and textile for the preparation of sheet-like articles in cosmetics, comprising
A) microemulsion concentrates according to claim 1, and
B) water, and also optionally
C) further cosmetic ingredients selected from the group consisting of anionic/nonionic, cationic and/or zwitterionic surfactants, and also optionally
D) further ingredients different to C).
